(19) United States
(12) Patent Application Publication
(10) Pub. No.: US 2022/0047434 A1
(43) Pub. Date: Feb. 17, 2022

(54) DISPOSABLE PROTECTION SHEET

(71) Applicant: G&E LIFE SOLUTIONS INC., Livermore, ME (US)

(72) Inventors: Jennifer LESSARD, Livermore, ME (US); Carl MALLOCH, Berkley, MA (US)

(21) Appl. No.: 17/395,857

(22) Filed: Aug. 6, 2021

Related U.S. Application Data
(60) Provisional application No. 63/065,647, filed on Aug. 14, 2020.

(51) Int. Cl. A61F 13/539; B32B 7/02; A61F 13/514

(52) U.S. Cl. CPC A61F 13/539; A61F 2013/15056; A61F 13/51456; B32B 7/02

(57) ABSTRACT

A multi-layered disposable protection sheet includes an upper portion and a lower portion including an upper absorbent layer, a middle soaker layer and a lower waterproof backing layer, the lower portion further including a cutout that when lifted, provides an opening to enable compatibility with a child safety belt system.

### CROSS REFERENCE TO RELATED APPLICATIONS

This application claims benefit from U.S. Provisional Patent Application Ser. No. 63/065,647, filed Aug. 14, 2020, which is hereby incorporated herein by reference in its entirety.

### BACKGROUND OF THE INVENTION

The present invention relates generally to chair covers, and more particularly to a disposable protection sheet.

In general, young children frequently soil while in, for example, their car seat, stroller, stationary seat. More specifically, they often spill, wet, and/or vomit while in these various seats. Historically, if the seat material on which the child rests gets wet or dirty because of soiling, one has to take the seat apart in order to remove the seat material to wash it. This process is time consuming and leaves one without a functional seat until the seat material is washed, dried, and put back on the seat.

What is needed is a way to protect the seat in a convenient, flexible and disposable manner.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In general, in one aspect, the invention features a disposable protection sheet including an upper portion, and a lower portion, the lower portion including a cutout that when lifted, provides an opening to enable compatibility with a safety belt system.

In another aspect, the invention features a multi-layered disposable protection sheet including an upper portion and a lower portion including an upper absorbent layer, a middle soaker layer and a lower waterproof backing layer, the lower portion further including a cutout that when lifted, provides an opening to enable compatibility with a child safety belt system.

Embodiments of the invention may include one or more of the following advantages.

A disposable protection sheet provides protection for infant and toddlers car seats, high chairs, booster seats, bouncers, and so forth.

The disposable protection sheet enables a custom fit to most any infant/child configuration.

The disposable protection sheet does not impede a fit of a baby, keeping it safe for all babies and toddlers. This custom fit also enables maximum protection from baby accidents, and protecting furniture while absorbing any mess.

In one implementation, disposable protection sheet includes a smell absorption feature to help absorb any smell until it can be removed.

The disposable protection sheet may be adapted to furniture needing protection from infant/child messes.

The details of one or more implementations of the present disclosure are set forth in the accompanying drawings and the description below. Other features and advantages of the present disclosure will be apparent from the description and drawings, and from the claims.

### BRIEF DESCRIPTION OF THE DRAWINGS

These and other features, aspects, and advantages of the present invention will become better understood with reference to the following description, appended claims, and accompanying drawings where:

FIG. 1 is exemplary child car seat with a disposable protection sheet.

FIG. 2 is exemplary child stroller with a disposable protection sheet.

FIG. 3 illustrates an exemplary stationary child seat with a disposable protection sheet.

FIG. 4 illustrates an exemplary disposable protection sheet.

FIG. 5 illustrates an exemplary multi-layered disposable protection sheet.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

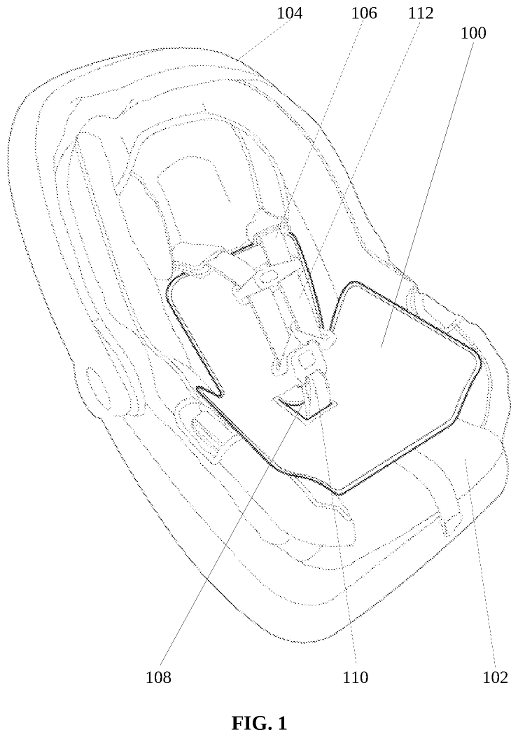

As shown in FIG. 1, an exemplary disposable protection sheet 100 is shown removably positioned on a cushion 102 of a car seat 104. In the example shown, the disposable protection sheet 100 is flexibly configured to be placed on the cushion 102 and protect a back, sides and bottom of a child placed in the car seat 104. In addition, the disposable protection sheet 100 is configured to enable use with a safety belt system 106 of the car seat 104. The disposable protection sheet 100 includes an opening 108 to enable a lower portion 110 of the safety seat system 106 to engage with an upper portion 112 of the safety seat system 106. In this example, the lower portion 110 and the upper portion 112 are sections of a safety belt.

Although shown with reference to the car seat 104, the disposable protection sheet 100 is easily configured to adapt to other forms of child seating.

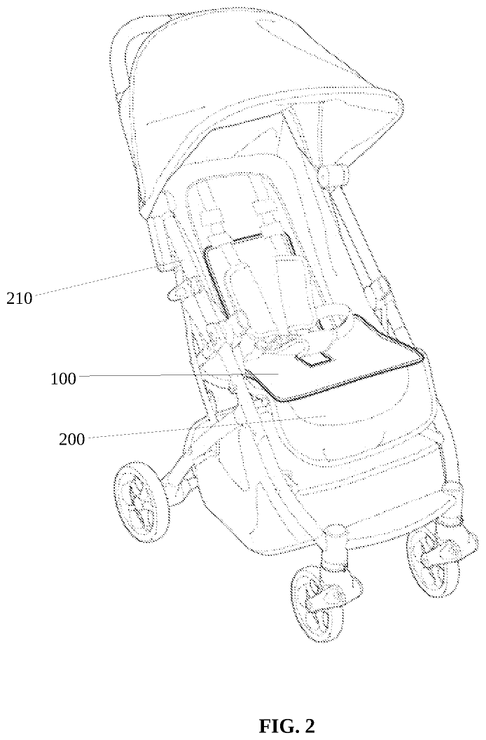

For example, FIG. 2 illustrates the disposable protection sheet 100 fitted in a seat 200 of an exemplary stroller 210.

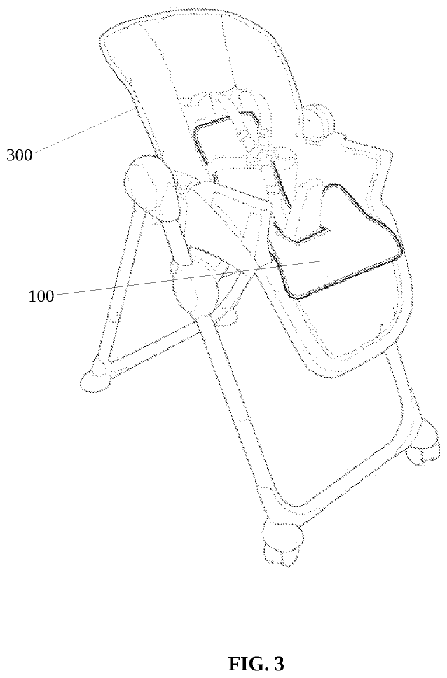

FIG. 3 illustrates the disposable protection sheet 100 an exemplary roller infant seat 300.

In summary, the disposable protection sheet 100 may be formed in various sizes and shapes to fit the need.

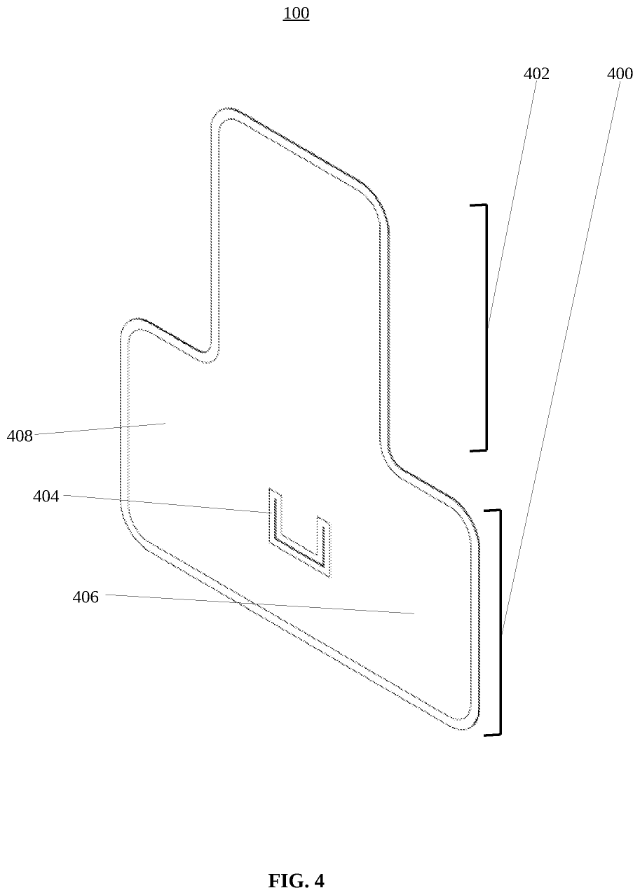

FIG. 4 illustrates a front prospective view of the disposable protection sheet 100 and includes a lower portion 400 and an upper portion 402. The lower portion 402 includes a cutout 404 that when lifted, provides an opening (e.g., opening 108 in FIG. 1) to enable compatibility with a safety belt system (e.g., safety belt system 106 in FIG. 1). The lower portion 402 includes left and right panel sections 406, 408 that enable the disposable protection sheet 100 to fit into the sides of a child car seat, stroller, and other types and varieties of infant seating.

In embodiments, the disposable protection sheet 100 may be constructed of various materials. For example, in one embodiment, the disposable protection sheet 100 includes a single layer while in another embodiment the disposable protection sheet 100 includes multiple layers.

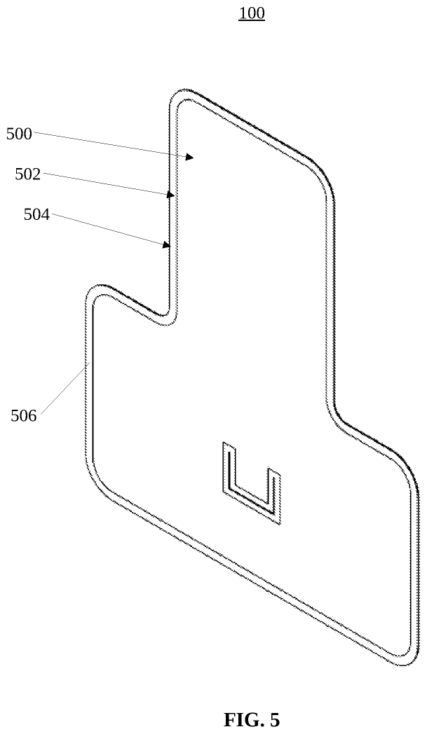

As shown in FIG. 5, in one specific implementation, the disposable protection sheet 100 includes a top absorbent layer 500, a middle soaker layer 502 and a third waterproof backing layer 504. The top absorbent layer 500 may constructed of a fabric that is flame retardant, e.g., cotton, cotton/poly blend, polyester. The middle soaker layer 502 may constructed of a material designed for absorbing liquids. The waterproof backing layer 504 may be constructed of a waterproof material, such as, nylon, polyurethane laminate, vinyl, and so forth. This layered construction enables liquid that seeps through the top layer 500 to be absorbed into the soaker layer 502 and then to stop penetrating at the waterproof backing layer 504. It also minimizes any odor associated with an unexpected child discharge.

A perimeter of the disposable protection sheet 100 includes a sown-in seam 506. The seam 506 provides at least three advantages. First, the seam 506 prevents any leakage of absorbed liquid. Second, the seam 506 provides a linkage of the top absorbent layer 500, the middle soaker layer 502 and the third waterproof backing layer 504. Third, the seam 506 aids in custom fitting.

The disposable protection sheet 100 is designed as a once and done protective sheet. In one embodiment, it is a seam sowed, heat sealed, and any type of sealing of the cutout material into a perimeter to aid in avoiding and minimizing leakage.

Since the disposable protection sheet 100 is not permanently affixed to a seat, once it is soiled it can be easily removed and sensibly disposed of and another fresh/clean disposable protection sheet 100 put in its place. In addition, the disposable protection sheet 100 is easily adapted to most any type of young child/infant seat; this enables a custom fit.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. A disposable protection sheet comprising:
an upper portion; and
a lower portion, the lower portion comprising a cutout that when lifted, provides an opening to enable compatibility with a safety belt system.

2. The disposable protection sheet of claim 1 wherein the lower portion further comprises:
a left panel section; and
a right panel section.

3. The disposable protection sheet of claim 2 wherein the upper portion and the lower portion comprise a first absorbent layer.

4. The disposable protection sheet of claim 3 wherein the first absorbent layer comprises a perimeter sealing.

5. The disposable protection sheet of claim 3 wherein the upper portion and the lower portion further comprise a second middle soaker layer.

6. The disposable protection sheet of claim 5 wherein the upper portion and the lower portion further comprise a third waterproof backing layer.

7. A multi-layered disposable protection sheet comprising:
an upper portion and a lower portion comprising an upper absorbent layer, a middle soaker layer and a lower waterproof backing layer,
the lower portion further comprising a cutout that when lifted, provides an opening to enable compatibility with a child safety belt system.

8. The multi-layered disposable protection sheet of claim 7 wherein the upper portion and the lower portion are constructed of a flexible material.

9. The multi-layered disposable protection sheet of claim 7 wherein the lower portion further comprises:
a flexible left panel section; and
a flexible right panel section.

10. The multi-layered disposable protection sheet of claim 7 wherein a perimeter of the upper portion and the lower portion are seam sealed.

11. The multi-layered disposable protection sheet of claim 7 wherein a perimeter of the upper portion and the lower portion are heat sealed.
